(19) United States
(12) Patent Application Publication
(10) Pub. No.: US 2018/0008450 A1
(43) Pub. Date: Jan. 11, 2018

(54) COLONOSCOPY BALL PILLOW

(71) Applicant: Arvind Movva, Moline, IL (US)

(72) Inventor: Arvind Movva, Moline, IL (US)

(21) Appl. No.: 15/641,890

(22) Filed: Jul. 5, 2017

Related U.S. Application Data
(60) Provisional application No. 62/358,315, filed on Jul. 5, 2016.

(51) Int. Cl. A61F 5/37; A61F 5/34; A61B 1/31

(52) U.S. Cl. CPC A61F 5/37; A61B 1/31; A61F 5/34

(57) ABSTRACT

The present invention includes methods of conducting a colonoscopy that includes positioning a ball pillow under the patient's left lateral lower abdomen while the patient is lying laterally until cecum is reached by the colonoscope. Also, the present invention includes methods of limiting looping of a colonscope during a colonoscopy that include positioning a ball pillow underneath a patient and/or applying a pressure to a patient's abdomen by securing a ball pillow between a patient and a surface.

### FIELD OF THE INVENTION

The present invention relates to methods and devices for conducting colonoscopies.

### BACKGROUND OF THE INVENTION

Colonoscopy is a procedure performed in hospitals, ambulatory surgery centers, and freestanding clinics to diagnose and evaluate the health status of the bowel and screen for colon cancer. Completing and appropriating inserting the scope is the most challenging part of the average colonoscopy. The goal of insertion is to reach an anatomic landmark called the cecum, which is the beginning of the large intestine (colon), allowing for complete evaluation and treatment of colonic disease states and detection and removal of pre-cancerous polyps.

During a colonoscopy, a scope is inserted into a patient's rectum and then advanced to the beginning of the colon (an area known as the cecum) in order to examine the lining of the large intestine. The efficiency and accuracy of this procedure is largely dependent on the ease with which the scope can be advanced. During the procedure, the colon may become over-distended or flopped in unnatural directions creating loops that hinder the advancement of the scope and resulting in patient discomfort, longer examination times, and potentially inaccurate or incomplete screenings.

During a colonoscopy, the colonoscope often loops (passively bends) in the more proximal portions of the colonoscope as turns in the patient's colon are navigated by the leading end of the colonoscope. Colonoscope looping is a common problem during colonoscopy that prolongs procedure time and occurs in up to 90% of all colonoscopies. Looping of the colonoscope is the primary cause of patient pain and increased procedure time while also increasing the risk of bowel perforation and splenic injury. Ancillary maneuvers such as abdominal pressure and patient position change are often used to correct looping, yet these maneuvers are applied variably, require extra personnel, are incompletely successful, and can increase procedure time and cost.

One known method of mitigating the problems caused by looping is to apply manual pressure. Applying manual pressure to the abdomen by an assistant (such as a nurse) is the most common technique. Techniques for applying this type of counter pressure have been well described in the literature, and include closed hand, open hand, and forearm methods. Each has its advantages and disadvantages. In the closed hand method, the assistant uses a fist at various points on the abdomen to apply counter pressure. The ability to feel loop formation with the fingertips is lost and the shoulder and neck muscles provide the necessary force. The forearm method places stress on the triceps and shoulder of the assistant and has the same lack of fingertip sensation to feel the formation of the loop. With the open handed method, where the senses of touch are most acute, the wrist is hyperextended. On average, pressure is apply for 6.3 minutes per colonoscopy. These problems lead to significant acute and chronic musculoskeletal injuries to those applying pressure—up to 36% of assistants have or have had acute or chronic soft tissue injuries related to applying counter pressure.

Re-positioning the patient is another method that is helpful in reducing looping. When difficulty reaching the cecum is encountered during a colonoscopy, patients are often re-positioned to the supine position. This position is associated with higher instance of aspiration (oral and gastric fluid entering the lungs) leading to complications like pneumonia and sometimes death.

A variety of wraps and pillows have also used to apply pressure to the abdomen. One such wrap, sold under the name ColoWrap, is applied to the patient's abdomen before beginning with sedation for the colonoscopy. It uses elastic cloth and bands to apply pressure. A peer-reviewed study showed a lack of effectiveness in the primary endpoints of decreased procedure time and need for manual abdominal pressure or position changes. A pillow, sold under then name N-Doe Pillow, is a reusable formed positioning device that uses two contact columns to apply abdominal pressure during colonoscopy. However, the N-Doe Pillow suffers from several drawbacks. No studies have been performed to show effectiveness.

The present invention overcomes one or more the drawback of known devices and techniques.

### SUMMARY OF THE INVENTION

The present invention includes methods of conducting a colonoscopy that includes positioning a ball pillow under the patient's left lateral lower abdomen while the patient is lying laterally until cecum is reached by the colonoscope. Also, the present invention includes methods of limiting looping of a colonscope during a colonoscopy that include positioning a ball pillow underneath a patient and/or applying a pressure to a patient's abdomen by securing a ball pillow between a patient and a surface.

### BRIEF DESCRIPTION OF THE DRAWINGS

FIGS. 1 to 3 show oblong ball pillows with consistent long dimensions, with varying short dimensions, so that the ratio of the long dimension to the short dimension is the smallest in FIG. 1, and the largest in FIG. 3, with the ratio in FIG. 2 therebetween.

Similar to FIGS. 1 to 3, FIGS. 4 to 6 show oblong ball pillows with consistent long dimensions that are longer than in FIGS. 1 to 3, with varying short dimensions, so that the ratio of the long dimension to the short dimension is the smallest in FIG. 4, and the largest in FIG. 6, with the ratio in FIG. 5 therebetween.

FIGS. 7 and 8 show spherical ball pillows with varying diameters.

FIGS. 9 and 10 show oblong ball pillows with rounded ends, like a rugby ball, with varying long dimensions and varying short dimensions.

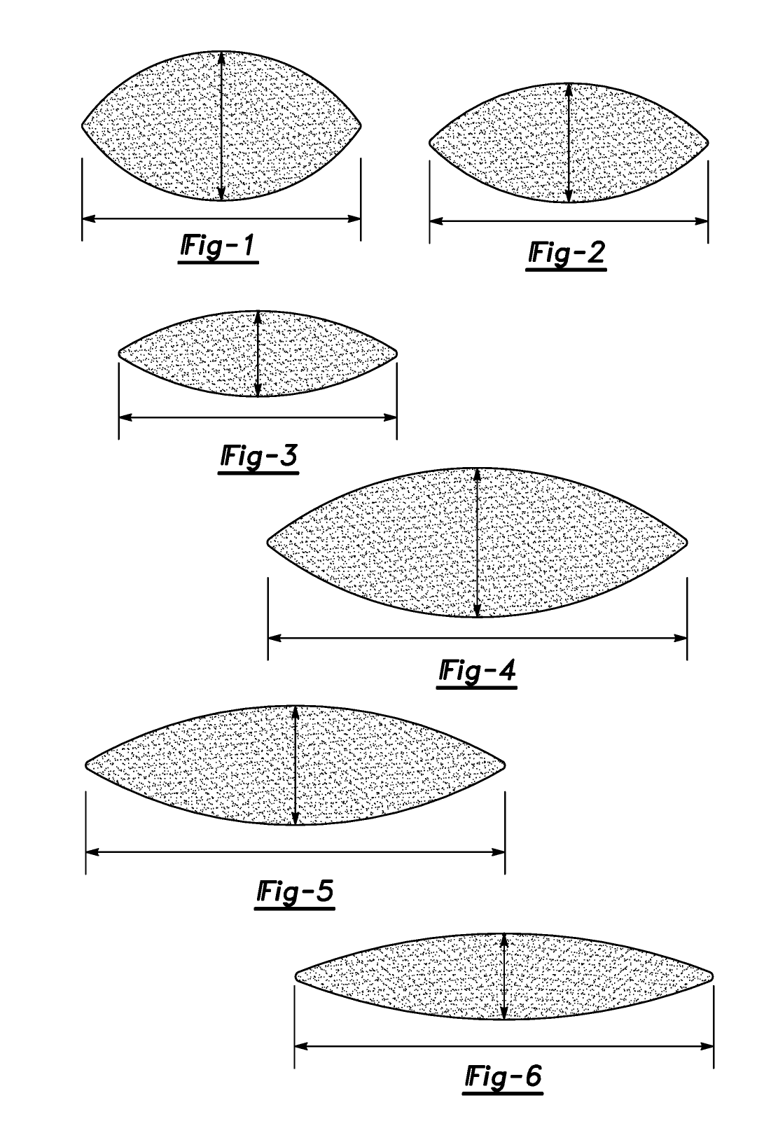

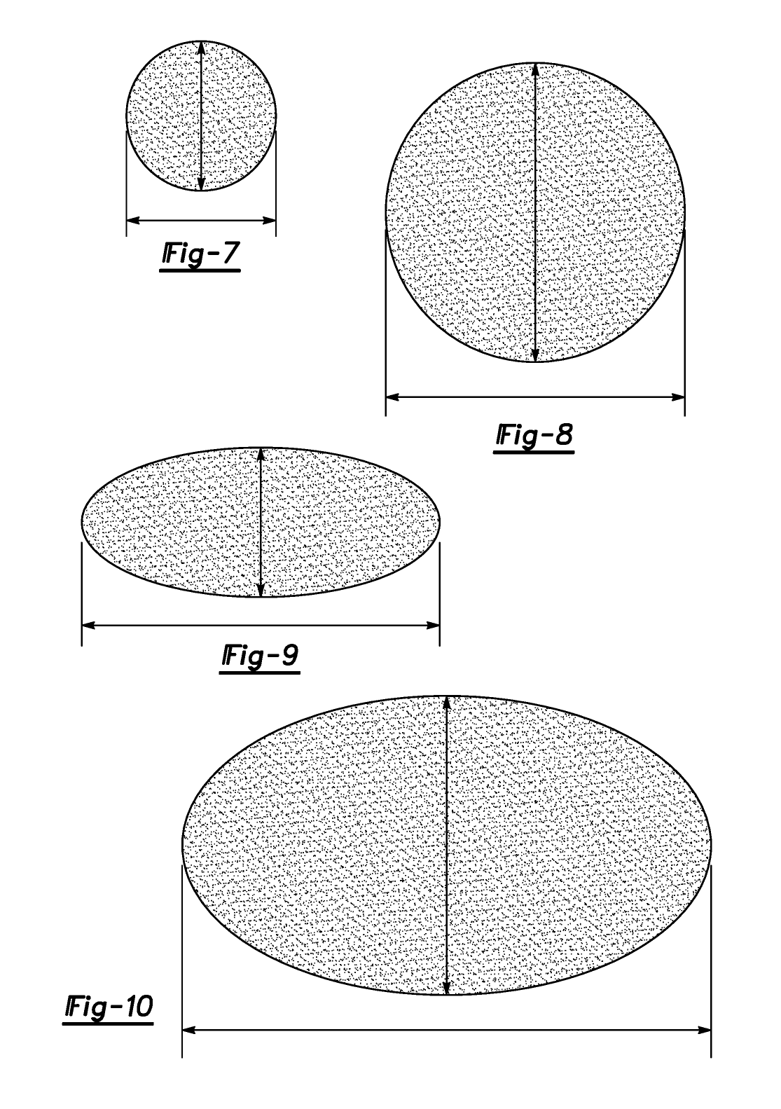

### DETAILED DESCRIPTION

The present invention includes positioning ball pillow under the patient's left lateral lower abdomen while the patient is lying on the patient's left side (i.e. in the lateral position). In general, the ball pillow is held in place between the patient and the surface on which the patient is lying, thus applying pressure on the patient's abdomen at the location of the ball pillow. Positioning of the ball pillow is preferably done before beginning the colonoscopy; that is, before the colonoscope is inserted into the rectum. The position of the ball pillow can be adjusted at any time. In one preferred embodiment, the ball pillow is removed once the cecum is successfully reached.

The ball pillow is preferably hollow and includes a port so that the ball pillow can be inflated (e.g. using atmospheric air) to achieve a variety of different interior pressures. Different inflation pressures result in different stiffnesses of the ball pillows and permit individualized treatment of patients. Some patients will benefit from a ball pillow with a lower inflation pressure, while others will benefit from a ball pillow with a higher inflation pressure. The overall stiffness of the inflatable ball pillow will depend on the stiffness of the material forming the ball pillow as well as the inflation pressure of the ball pillow. Foam ball pillows of varying softness or hardness may also be used, however inflatable ball pillows are preferred because the stiffness can be selected after manufacture. Indeed, the stiffness of the inflatable ball pillows could be adjusted after positioning with a hand pump; similar to a blood pressure cuff. The preferred inflation pressure of the ball pillow ranges from about 14 psi (e.g. 1 atm) up to about 100 psi, although other inflation pressures are also contemplated.

The ball pillow is preferably sized to provide the most comfort to the patient. Patients with a high body-mass index (BMI) and/or waist circumference will benefit from a smaller sized ball pillow compared to relatively larger sized ball pillows. Conversely, patients with a low BMI and/or waist circumference will benefit from a larger sized ball pillow.

The overall stiffness of the ball pillow (that is, material stiffness and inflation pressure) is preferably selected to provide the most comfort to the patient while accomplishing the goal of preventing looping. Patients with a high BMI and/or waist circumference are thought to benefit from a softer ball pillow (generally lower inflation pressure) compared to relatively harder ball pillows. Conversely, patients with a low BMI and/or waist circumference are thought to prefer a harder ball pillow.

Likewise, the shape of the ball pillow is preferably selected to provide the most comfort to the patient. For example, spherical ball pillows are thought to be beneficial for patients with low BMI while oblong (e.g. football ball shaped) ball pillows are thought to be beneficial for patients with a high BMI.

Of course, all three characteristics of the ball pillows may be optimized based the height, waist circumference, and BMI of the patient. For example, a large, spherical, hard ball pillow might be preferred for a patient with a low BMI, while a small, oblong, soft ball pillow might be preferred for a patient with a high BMI. Of course, for patients with a middling BMI, a medium sized, slightly oblong, and medium stiffness ball pillow might be preferred. In one set of embodiments, all three of these characteristics move in conjunction with each other along the continuum between the extremes of each a characteristic. That is, as the shape moves from spherical to more elongated, the size moves from large to small, and the stiffness moves from hard to soft. Also, one or two characteristics may be constant while the other characteristic move along the continuum. For example, a single size ball pillow may be used, but the shape and/or stiffness varied for patients having different heights, waist circumferences, and/or BMIs.

While each patient will be different because individualized physiology, in general, a low BMI refers to BMIs of about 25 and less, a high BMI refers to BMIs of about 30 and more, and a middling BMI refers to BMIs therebetween.

In one preferred embodiment, the ball pillow is oblong shaped, such as an American football ball or a rugby ball, with a long dimension of about 9 inches and a short dimension of about 5 inches. The range of preferred embodiments of oblong shaped pillows include those with a long dimension between about 4 and 14 inches, and those with a short dimension between about 1 and 10 inches. The preferred range of ratios of long dimension to short dimension (for oblong pillows) is about 1.5 to 2.1. Of course, spherical pillows are also contemplated.

Preferably, the ball pillow is a single use device as to avoid contamination and spread of infectious materials like blood, stool, and urine encountered during a colonoscopy.

The use of a ball pillow has several advantages over prior techniques. It is believed that the ball pillow will significantly reduce the need for any manual abdominal pressure, significantly reduce the need for any re-positioning of the patient, reduces the risk of musculoskeletal injury to assistants, all the while significantly reducing the time and complexity of completing a successful colonoscopy.

The ball pillow preferably deforms at least a little bit as the patient's body is positioned against it, allowing the ball to partially conform to the patient's body and thus place diffuse pressure and avoiding placing too much pressure on any one particular area of the patient's body.

Also, the ball pillow places pressure in only one location at a time; that is, on the lower left side of the patient's abdomen when the patient is lying on the patient's left side. This simplifies the positioning and use of the ball pillow of compared to other solutions. The ball has just one positioning parameter; that is, it is positioned in a single step. In contrast, other solutions require that a first portion of a device be positioned against a first location on the patient's abdomen and than a second portion of the device is positioned on another location of the patient's abdomen. This may involve adjusting the patient's position in order to achieve an acceptable amount of pressure to prevent looping.

Further, the ball pillow can be easily removed once the cecum is reached during the colonoscopy, and the risk of complications due to compression of the abdomen can be reduced (such as patient discomfort and aspiration). The reduction in overall procedure time leads to less sedative needing to be administered to the patient, which in turn reduces the risk of the procedure to the patient. The ball pillow is effective across different size (height, waist circumferences, and body mass index) of patients. All of this significantly reduces the difficulty of performing a colonoscopy by endoscopist, while the single use nature of the ball pillow decreases risk of contamination and passing infection from patient to patient.

In addition to positioning of the ball pillow, the method of the present invention may include other steps that occur before or after the placement of the ball pillow such as timing of sedation, initially positioning the patient on the table (typically on the patients left side), positioning the ball pillow, re-positioning the ball pillow, leaning the patient onto the ball pillow, removing the ball pillow, applying manual abdominal pressure in conjunction with the ball pillow, etc. All steps related to the ball pillow placement can be done before, during or after the colonoscopy or the initiation of the insertion of the colonoscope.

In addition to steps related to the placement of the ball pillow, the method may further include steps related to conducting the colonoscopy such inserting the scope, advancing the scope to the cecum, further advancing the scope past the cecum, performing biopsies, removing polyps, removing the scope, etc.

It will be further appreciated that functions or structures of a plurality of components or steps may be combined into a single component or step, or the functions or structures of one-step or component may be split among plural steps or components. The present invention contemplates all of these combinations. Unless stated otherwise, dimensions and geometries of the various structures depicted herein are not intended to be restrictive of the invention, and other dimensions or geometries are possible. Plural structural components or steps can be provided by a single integrated structure or step. Alternatively, a single integrated structure or step might be divided into separate plural components or steps. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A method of limiting looping of a colonoscope during a colonoscopy, comprising:
positioning a ball pillow against a patient's lower left abdomen when the patient is lying on the patient's left side on a surface; and
initiating a colonoscopy of the patient.

2. The method of claim 1 wherein the ball pillow is an inflatable ball pillow.

3. The method of claim 2 further comprising adjusting an inflation pressure of the inflatable ball pillow after the positioning step.

4. The method of claim 2 wherein the inflatable ball pillow is oblong in shape.

5. The method of claim 3 further comprising sedating the patient prior to positioning step.

6. The method of claim 4 further comprising, after initiating the colonoscopy, advancing colonoscope to the patient's cecum.

7. The method of claim 5 further comprising removing the inflatable ball pillow after the colonscope has been advanced to the patient's cecum.

8. A method of limiting looping of a colonoscope during a colonoscopy, comprising:
applying pressure to a patient's lower left abdomen when the patient is lying on the patient's left side by securing a ball pillow between the patient and a surface.

9. The method of claim 8 wherein the ball pillow is an inflatable ball pillow.

10. The method of claim 9 further comprising adjusting an inflation pressure of the inflatable ball pillow after the applying pressure step has been initiated.

11. The method of claim 8 further comprising initiating a colonoscopy by inserting a colonoscope into the patient.

12. The method of claim 11 further comprising advancing the colonoscope toward the cecum and removing pressure from the patient's lower left abdomen by removing the inflatable ball pillow after the cecum has been reached by the colonoscope.

13. A method of conducting a colonoscopy, comprising:
applying pressure to a patient's lower left abdomen when the patient is lying on the patient's left side by securing a ball pillow between the patient and a surface;
initiating a colonoscopy by inserting a colonoscope into the patient;
advancing the colonoscope toward the cecum; and
removing pressure from the patient's lower left abdomen by removing the ball pillow after the cecum has been reached by the colonoscope.
